(19) United States
(12) Patent Application Publication
(10) Pub. No.: US 2002/0193813 A1
(43) Pub. Date: Dec. 19, 2002

(54) Hydrogel filament vaso-occlusive device

(75) Inventors: Richard A. Helkowski, Redwood City, CA (US); Christopher G.M. Ken, San Mateo, CA (US); Tina J. Patel, San Carlos, CA (US)

Correspondence Address: BANNER & WITCOFF, 1001 G STREET N W, SUITE 1100, WASHINGTON, DC, 20001, US

(73) Assignee: Concentric Medical, Mountain View, CA

(21) Appl. No.: 10/138,535

(22) Filed: May 6, 2002

Related U.S. Application Data
(60) Provisional application No. 60/288,458, filed on May 4, 2001.

(51) Int. Cl. A61M 29/00

(52) U.S. Cl. 606/151; 606/191

(57) ABSTRACT

Methods and an apparatus for treating abnormal blood flow. The apparatus comprises a vaso-occlusive device of a hydratable filament comprising extruded polyacrylonitrile for implantation in a patient at a site of abnormal blood flow. The device treats ruptured blood vessels, aneurysms, arterio venus malformations (AVMs), fistulas and benign and malignant tumors. The methods include a method of making the vaso-occlusive device and methods of treating patients having abnormal blood flow by implanting the device at a site of abnormal blood flow.

### CROSS-REFERENCE TO RELATED APPLICATION

This application claims benefit under 37 CFR §1.78 of provisional application No. 60/288,458, filed May 4, 2001. The full disclosure of the application is incorporated hereby by reference.

### FIELD OF THE INVENTION

The present invention relates to medical devices and methods for vaso-occlusion.

### BACKGROUND OF THE INVENTION

Ruptured blood vessels in the brain cause an acute condition known as hemorrhagic stroke. Ruptures or strokes can occur with a number of vascular abnormalities including arterio venous malformation (AVM), aneurysm (a ballooning of the arterial wall), fistula, or a burst blood vessel. In addition, abnormal vasculature is generated in the process of tumor growth and tumors including brain tumors are highly vascularized entities requiring larger than normal blood flow to sustain the tumor.

Endovascular therapy for vaso-occlusion has included injectable agents, balloon-type occlusive devices, and mechanical vaso-occlusive devices such as metal coils. A description of these agents and devices is included in the background section of U.S. Pat. No. 4,994,069.

Currently, coils for aneurysms and polyvinyl alcohol (PVA) particles for AVMs are FDA approved preventative therapies. Cyanoacrylate glue for AVMs is also proposed and pending approval.

Over 400,000 persons worldwide, and 125,000 persons in the U.S. annually experience some form of hemorrhagic stroke or blood vessel rupture in the brain. Many presently known and used devices for implantation to treat abnormal blood flow fall short of efficacy desired. As a result, a need exists in the medical community, particularly in the field of interventional neurology, for devices and/or agents that can be effectively used in interventional neurology treatments for strokes and tumors.

### SUMMARY OF THE INVENTION

The invention provides a vaso-occlusive device for implantation into the vasculature of a patient to occlude blood flow comprising:

a hydratable filament comprising extruded polyacrylonitrile.

The device can further comprise an effective amount of a bioactive agent incorporated into the filament during extrusion or subsequent hydration of the filament resulting from extrusion; wherein said bioactive agent acts in the patient to provide a biological activity at a site of implantation of the vaso-occlusive device.

The bioactive agent can promote an activity at the site of implantation selected from the group consisting of occludes blood flow, adheres the device at the site, rebuilds a damaged vascular wall, regresses or inhibits capillary dilation, regresses or inhibits venus malformation, and regresses or inhibits tumor growth at or near the implantation site.

The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The device can further comprise a radio pacifier.

The radio pacifier can comprise a contrast agent or a metal powder.

The invention also provides a method of making a vaso-occlusive device comprising extruding a hydratable filament comprising polyacrylonitrile.

The method can further comprise dissolving polyacrylonitrile in DMSO, extruding the DMSO solution into isopropyl alcohol, and forming a filament in the alcohol.

The method can still further comprise evaporating the alcohol or removing the filament from the alcohol to dry.

The method can also further comprise hydrating the dehydrated filament for storage or delivery into a patient.

The device can further comprise a bioactive agent integrated into the extruded product. Integrating the bioactive agent into the extruded product can be accomplished either during extrusion or after extrusion. Integrating the bioactive agent can be accomplished after extrusion, and the post-extrusion integrating can be selected from the acts consisting of coating, dipping, jacketing, spraying, weaving, braiding, spinning, ion implantation, vapor deposition and plasma deposition. Integrating the bioactive agent can be accomplished during extrusion, and the integrating is accomplished by placing the bioactive agent into a solvent used to dissolve the polyacrylonitrile.

The invention provides a method of treating a patient having abnormal blood flow at a site in the patient comprising injecting into the patient at the site of abnormal blood flow a material comprising an extruded hydratable filament comprising polyacrylonitrile.

The method can further comprise coating the injectable filament with a bioactive agent, or integrating a bioactive agent into the injectable filament.

Coating or integrating can comprise a process selected from the group consisting of coating, dipping, jacketing, spraying, weaving, braiding, spinning, ion implantation, vapor deposition and plasma deposition.

### BRIEF DESCRIPTION OF THE DRAWINGS

FIG. 1A shows a filament before implantation in a patient; FIG. 1B shows a filament after implantation in a patient.

### DETAILED DESCRIPTION OF THE DRAWINGS

The following embodiments and examples are offered by way of illustration and not by way of limitation.

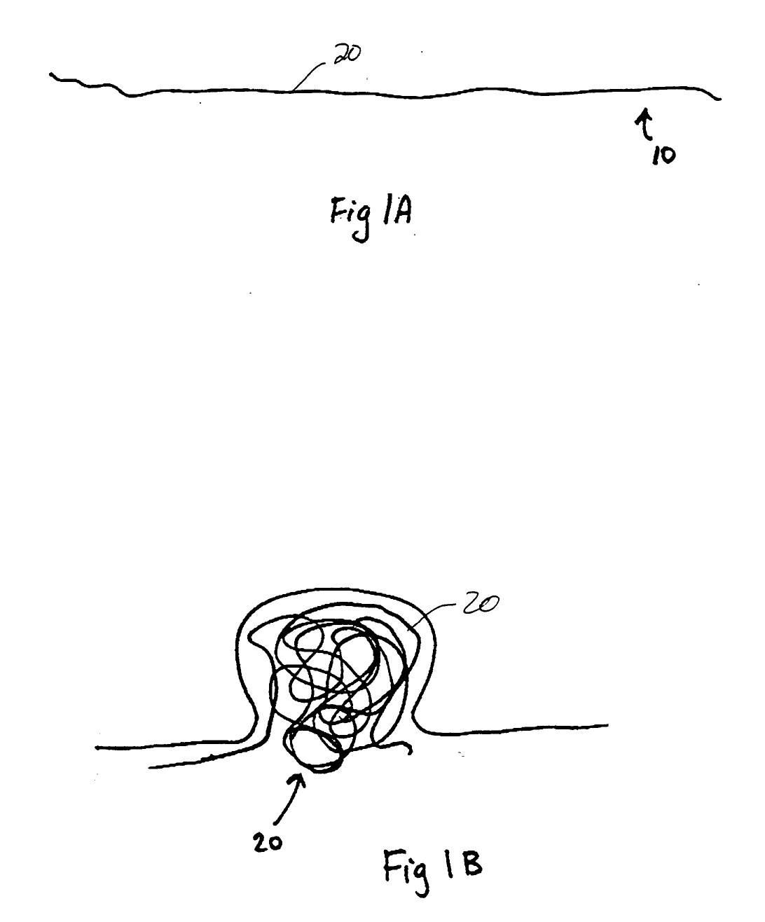

Turning first to the figures, FIGS. 1A and 1B depict a vaso-occlusive device 10 according to the present invention. The vaso-occlusive device 10 includes a filament 20. FIG. 1A illustrates the filament 20 in an elongated, pre-implantation shape. In one embodiment, the filament 20 is formed by extrusion as discussed below. FIG. 1B depicts the filament 20 after implantation at a site of abnormal blood flow. In FIG. 1B, the filament 20 has assumed an implanted or vaso-occlusive shape.

The filament 20 of the vaso-occlusive device 10 includes a hydratable filament comprised of extruded polyacrylonitrile. Polyacrylonitrile can be made as described in Stoy et al U.S. Pat. No. 4,943,618, Stoy et al U.S. Pat. No. 4,337,327, Stoy et al U.S. Pat. No. 4,370,451, Zimmerman et al U.S. Pat. No. 4,331,781, Stoy et al U.S. Pat. No. 4,369,294, Stoy et al U.S. Pat. No. 4,420,589, and Stoy et al U.S. Pat. No. 4,379,874.

Other polymers that can be used to form the filament 20 include a polymer or polymers selected from the group consisting of polyacrylamide (PAAM), poly (Nisopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(P-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP) Polyglycolic Lactic Acid (PGLA), a copolymer, and a blend of two or more polymers. The PGLA can be formed by mixing PGA: PLA with ratios of 99.9: 0.1 to 50:50.

The present invention also includes a method for making the filament 20 that forms at least a portion of the device 10. This method of making filament 20 includes the steps of dissolving the polyacrylonitrile in DMSO and extruding it into an alcohol bath, whereupon the polyacrylonitrile solution forms the filament 20. The filament 20 can then be removed from the alcohol (e.g. isopropyl or like alcohol) and allowed to dry. Prior to implantation in a patient the filament 20 can be hydrated. Alternatively, the filament 20 can be stored under hydrating conditions.

As discussed below, after the filament 20 has been formed to a predetermine length, the filament 20 can then be implanted in the patient. The hydrated filament 20 can be injected or delivered in a delivery tool to a site of abnormal blood flow in the patient. After or during the implantation step, the hydrated filament 20 forms a vaso-occlusive filamentous mass and occludes abnormal blood flow as shown in FIG. 1B.

Extrusion of the filament 20 can be accomplished by standard methods and processes of extrusion known in the art. The hydrant used in the present invention can comprise water or a solution that comprises water and other elements. The method of the present invention forms the injectable filament 20. The quality of the filament 20 is derived from the stringy filamentous quality of the resulting extruded product, and the fact that it is extruded with the needle tip fully in alcohol. The filament 20 is ideal for delivery to a site of abnormal blood flow for occlusion purposes. Delivery of the filament 20 can be accomplished by standard process known in the art for implanting a vaso-occlusive device, e.g. a catheter or other suitable lumen with a pusher or pressure application system and the like can be used.

In an embodiment, the filament(s) 20 is delivered to the surgeon, other practitioner or attendant in pre-cut lengths. In this embodiment, each filament is cut or formed to a predetermined length. For example, the length of the filament 20 of the vaso-occlusive device 10 as it is delivered can be in the range from about 1 mm to about 5 meters. In a preferred embodiment, the pre-cut lengths of the filament(s) 20 of the vaso-occlusive device 10 for delivery to the patient can be in a range from about 1 mm to about 10 mm. In an embodiment, the dimensions of the device 10 can be from about 0.125 mm to about 12.50 mm, or the outside diameter of objects suitable for passing through a delivery device to a site of abnormal bleeding. The diameter of the vaso-occlusive device 10 once it is delivered and after it has assumed its vaso-occluding shape (FIG. 1B) can be in a range from about 1 mm to about 50 mm.

Another embodiment of the vaso-occlusive device 10 as described further comprises a bioactive agent integrated with the polyacrylonitrile material. The integration of the bioactive agent with the polyacrylonitrile material can be accomplished in a first embodiment by mixing the bioactive agent (or agents, if more than one bioactive agent is combined for delivery) with the polyacrylonitrile material before forming the filament. In a second embodiment the integration of the bioactive agent with the polyacrylonitrile material, or, also by example, contacting the polyacrylonitrile with the agents (e.g. a powder or solution of the agent) in the alcohol during the extrusion. In addition, the hydrating solution might also comprise one or more bioactive agents for contacting the dehydrated filament and being absorbed into the absorbent filamentous material. Alternatively, the bioactive agent can be coated onto the dehydrated or hydrated filament, for example by coating, dipping, jacketing, spraying, weaving, braiding, spinning, ion implantation, vapor deposition or plasma deposition of the bioactive material onto or into the filament.

U.S. Pat. No. 5,808,012 describes a process by which proteins and other bioactive agents can be incorporated into a polymer during a forming process such as extrusion, molding, casting. The process described can be used in the present invention to incorporate one or more of the above-discussed proteins or other bioactive agents into one or more of the above-discussed polymers.

U.S. Pat. No. 6,184,348 describes production of novel polymers using recombinant techniques, and also integration of bioactive agents potentially useful at a site of implantation in the patient. U.S. Pat. No. 6,184,348 also describes spinning applicable here as a way to incorporate a bioactive agent. These methods could be used to form the above-discussed compounds.

The bioactive agents used with the filament 20 can be an agent that promotes any biological activity desired at the site of abnormal blood flow. Some possible desired biological activities can include (but are not limited to) for example, occluding blood flow, adhering the device at the site of implantation, building a damaged vascular wall, regressing capillary dilation, inhibiting capillary dilation, regressing an AVM, inhibiting an AVM, regressing tumor growth, or inhibiting tumor growth, to name a few but not all of the possible or desired biological activities that could be present in any given selected bioactive agent.

The above-discussed bioactive agent can, accordingly, be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The amount of the bioactive agent used will preferably be an amount sufficient for the agent to be effective at the site of implantation for the biological activity expected from the agent. What would be an effective amount for any given agent or agents can be determined on an agent-by-agent basis, taking into account standard, known parameters of any given bioactive agents such as potency, available concentration, and volume of space within the patient to be targeted for the desired effect. Efficacy and proper dosage can be determined by routine assay specific for the bioactive agent selected using for example standard known assays provided in well known frequently used laboratory assay and protocol manuals for identifying activity and quantifying potency of molecules and cells.

The vaso-occlusive device 10 can also comprise a radio pacifier. The radio pacifier can comprise an agent that provides visibility of the device under X-ray or other imaging technology such as, for example, CT scans, MRIs and flouroscopy. In one embodiment, the radio pacifier includes a gadolinium-based MRI contrast agent. These agents can include, but are not limited to, Gadopentetate, Gadopentetate dimeglumine (Gd DTPA or Magnevist (R)), Gadoteridol (Gd HP-DO3A or ProHance (R)), Gadodiamide (Gd DTPA-BMA or Omniscan (R)), Gadoversetamide (Gd DTPA-BMEA or OptiMARK (R)), Gd-DOTA (Magnevist (R) or Dotarem (R)), Gd-DTPA labeled albumin, and Gd-DTPA labeled dextran.

In additional embodiments, the radio pacifier can comprise, for example, a contrast media or a metal powder, but is not limited to these items. The metal powder can be, for example, titanium, tungsten, gold, barium sulfate, bismuth or tantalum powder. The radio pacifier can be integrated into the dissolved polyacrylonitrile before extrusion, thus resulting in an extruded filament 20 comprising the radio pacifier. Alternatively, the radio pacifier can be coated or integrated into the dehydrated or hydrated filament, for example by coating, dipping, jacketing, spraying, weaving, braiding, spinning, ion implantation, vapor deposition or plasma deposition of the radio pacifier onto or into the filament. Further alternatively, the radio pacifier can be present in a hydration solution and can be absorbed into the filament as it hydrates. By including such a radio pacifier in/on the device 10, the device 10 can be monitored and detected once inside the patient.

As mentioned above, the present invention also includes a method of making the vaso-occlusive devices 10 described herein. The method comprises extruding the hydratable filament 20 comprising polyacrylonitrile, as described above. The process can further comprises integrating the bioactive agent into the extruded product. Integrating the bioactive agent into the extruded product can be accomplished either during extrusion or after extrusion. Thus, the bioactive agent can be mixed with the polyacrylonitrile and integrated into the resulting filament as the polyacrylonitrile is extruded into an alcohol bath. After extrusion, the filament 20 can be coated with a bioactive agent, e.g. by coating, dipping, jacketing, spraying, weaving, braiding, spinning, ion implantation, vapor deposition or plasma deposition. The bioactive agent may also be combined in the hydration solution and absorbed by the filament 20 as it hydrates. Similarly, and as described above, a radio pacifier can be incorporated into the filament 20 for detection of the device in the patient after implantation.

The invention also provides a method of treating a patient having abnormal blood flow at a site in the patient body comprising injecting into the patient at the site of abnormal blood flow the device 10 formed of a material comprising the extruded hydrated filament 10 comprising polyacrylonitrile. To further and possibly more effectively treat the patient, the method can further comprise providing also a bio active agent (or more than one bioactive agent), such as those agent or agents described herein, integrated with or coating the filament. Once placed at the site of implantation the bioactive agent provides an expected biological activity at the site. To practice the method of treating a patient, the filament 20 is formed and hydrated either before or during delivery to the site of abnormal blood flow in the patient.

All publications, patents and patent applications cited in this specification are herein incorporated by reference as if each individual publication, patent or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A vaso-occlusive device for implantation into the vasculature of a patient to occlude blood flow comprising:
a hydratable filament comprising extruded polyacrylonitrile.

2. A vaso-occlusive device as in claim 1, further comprising a bioactive agent incorporated into the filament during extrusion or subsequent hydration of the filament resulting from extrusion; wherein said bioactive agent acts in the patient to provide a biological activity at a site of implantation of the vaso-occlusive device.

3. A vaso-occlusive device as in claim 2, wherein the bioactive agent promotes an activity at the site of implantation selected from the group consisting of occludes blood flow, adheres the device at the site, rebuilds a damaged vascular wall, regresses or inhibits capillary dilation, regresses or inhibits venus malformation, and regresses or inhibits tumor growth at or near the implantation site.

4. A vaso-occlusive device as in claim 2, wherein the bioactive agent is selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

5. A vaso-occlusive device as in claim 1, further comprising a radio pacifier.

6. A vaso-occlusive device as in claim 5, wherein the radio pacifier comprises a contrast agent or a metal powder.

7. A method of making a vaso-occlusive device comprising a step of extruding a hydratable filament comprising polyacrylonitrile.

8. A method as in claim 7, further comprising steps of: dissolving polyacrylonitrile in DMSO, extruding the DMSO solution into isopropyl alcohol, and forming a filament in the alcohol.

9. A method as in claim 8, further comprising evaporating the alcohol or removing the filament from the alcohol to dry.

10. A method as in claim 9, further comprising hydrating the dehydrated filament for storage or delivery into a patient.

11. A method of making a vaso-occlusive device as in claim 7, further comprising a step of integrating a bioactive agent into the extruded product.

12. A method of making a vaso-occlusive device as in claim 11, wherein integrating the bioactive agent into the extruded product is accomplished either during extrusion or after extrusion.

13. A method as in claim 12, wherein integrating the bioactive agent is accomplished after extrusion, and the post-extrusion integrating is selected from the acts consisting of coating, dipping, jacketing, spraying, weaving, braiding, spinning, ion implantation, vapor deposition and plasma deposition.

14. A method as in claim 12, wherein integrating the bioactive agent is accomplished during extrusion, and the integrating is accomplished by placing the bioactive agent into a solvent used to dissolve the polyacrylonitrile.

15. A method of treating a patient having abnormal blood flow at a site in the patient comprising a step of:
injecting into the patient at the site of abnormal blood flow a material comprising an extruded hydrated filament comprising polyacrylonitrile.

16. A method of treating a patient as in claim 15, further comprising coating the filament with a bioactive agent, or integrating a bioactive agent into the filament.

17. A method as in claim 15, wherein coating or integrating comprises a process selected from the group consisting of coating, dipping, jacketing, spraying, weaving, braiding, spinning, ion implantation, vapor deposition and plasma deposition.

18. A vaso-occlusive device as in claim 2, wherein the bioactive agent is PGLA.

19. A vaso-occlusive device as in claim 18, wherein the PGLA is formed by mixing PGA:PLA in ratios ranging from about 99.9:0.1 to about 50:50.

20. A method as in claim 7, further comprising dissolving polyacrylonitrile in sodium thiocyanate, extruding the sodium thiocyanate into isopropyl alcohol, and forming a filament in the alcohol.

21. A method as in claim 20, further comprising evaporating the alcohol or removing the filament from the alcohol to dry.

22. A method as in claim 21, further comprising hydrating the dehydrated filament for storage or delivery into a patient.
